(19) United States
(12) Patent Application Publication
(10) Pub. No.: US 2020/0179226 A1
(43) Pub. Date: Jun. 11, 2020

(54) DRY INTRAVENOUS POWDER COMPOSITIONS AND USES THEREOF

(71) Applicant: Matthew Balla, Bloomington, IN (US)

(72) Inventor: Matthew Balla, Bloomington, IN (US)

(21) Appl. No.: 16/211,908

(22) Filed: Dec. 6, 2018

(51) Int. Cl. A61J 1/20; A61J 1/10; A61K 9/00; A61K 9/19; A61K 9/14; A61K 31/7004; A61K 33/14

(52) U.S. Cl. CPC A61J 1/2093; A61J 1/10; A61K 9/0019; A61K 33/14; A61K 9/14; A61K 31/7004; A61K 9/19

(57) ABSTRACT

A pre-loaded sterile container in a storage configuration includes a sterile container that defines a recess with a dry powder composition therein. The dry powder composition includes sterile electrolytes and/or dextrose. The pre-loaded container is configured to allow for the introduction of a prescribed amount of sterile water in the recess for agitation with the dry powder composition when needed by a medical patient. After introduction of the sterile water, the pre-loaded container is in a patient dispensing configuration and contains an IV fluid. The dry powder composition in the sterile container results when an IV fluid in the recess of the sterile container undergoes one or more processes such as sterile filtration, spray drying, lyophilization, and/or gamma radiation. The pre-loaded sterile containers are lightweight and have a long shelf-life as compared to containers or bags that simply hold an IV fluid.

### TECHNICAL FIELD

The present disclosure generally relates to providing dry powder compositions in sterile containers, and more particularly, but not exclusively to reconstituting the dry powder compositions by adding sterile water for injection to the sterile containers that contain the dry powder compositions therein to be used as large volume parenteral infusions or bulk IV fluids.

### BACKGROUND

Traditional healthcare practice involves hospitals, providers and clinics purchasing and using pre-manufactured intravenous (IV) fluids. Some of these IV fluids include, but are not limited to, dextrose 5%/water 1000 mL, dextrose 5%/0.9% sodium chloride 1000 mL, and dextrose 5%/0.45% sodium chloride+20 mEq/Liter KCl 1000 mL. These IV fluids are used to hydrate patients, maintain blood pressure as well as provide infusion vehicles for other medications. IV fluids are ubiquitous to the healthcare industry.

There are multiple challenges associated with IV fluids. The various combinations and types of fluids require tremendous shipping, supply chain, storage and logistical challenges. Despite their simplicity, IV fluids remain one of the highest volume and expensive items for hospitals.

One of the challenges associated with IV fluids in bags includes shipping costs. For example, each case of IV fluids typically weighs about 13-14 Kg and typically contains 12 bags containing IV fluid per case. As one can appreciate, the shipping costs correspond to the weight of the case so a heavier case will typically cost more to ship or transport than a lighter case. Moreover, the amount of energy used to transport these heavy bags and cases is higher than that required to transport a similar number of lighter bags.

Another challenge associated with IV fluids in bags includes the supply chain and storage. For example, end use facilities or institutions including but not limited to hospitals, nursing homes, recovery centers, and military installations require large footprint storage facilities and space for storing IV fluids in bags. The large storage facilities in hospitals, nursing homes, and recovery centers require space use of these institutions that could alternatively be utilized for more value-added patient care and/or patient beds wherein in either situation the patients will correspondingly pay for these services which provides a revenue source for the institution. Additionally, many hospitals use automated medicated dispensing cabinets to store the IV fluids. These automated medicated dispensing cabinets are costly to buy or lease. The large storage facilities in military institutions are also problematic in that the military and similarly first responders place storage space and supply weight at a premium. Additionally, the heavy cases of IV fluids are typically moved by employees of the institution and/or military who can be easily injured while moving the heavy cases.

Other challenges associated with IV fluids in bags include logistics and transportation. Many healthcare facilities carry up to 20 different types of IV fluids to meet the various patient care needs which result in logistics issues. This requires time, effort, and labor to maintain a supply of the IV fluids as well as ordering replenishment of the IV fluids as needed, and storage of these large and bulky bags. Transportation challenges associated with IV fluids starts at the manufacturing facilities. Manufacturers add the shipping costs to the purchase price of these IV fluids, adding to the expense of patient care. Furthermore, once the fluids are procured by hospitals, supply chain employees are needed to distribute IV fluids within hospitals and clinics. These additional employees increase labor, which also adds to the cost of healthcare.

Furthermore, natural disasters such as hurricanes and the like can negatively impact the overall supply of IV fluids. As one can appreciate, natural disasters can destroy facilities where these IV fluids are manufactured and/or stored. Moreover, due to the size and weight of IV bags, first responders may be limited in the variety of IV fluids they can transport when responding to those in need that are affected by the natural disasters.

Therefore, further contributions in this area of technology are needed to improve IV fluids. Therefore, there remains a significant need for the apparatuses, methods and systems disclosed herein.

### SUMMARY OF THE DISCLOSURE

Unique apparatuses, systems, and methods are disclosed for reducing the cost of patient care. These apparatuses, systems, and methods greatly reduce the risk associated with supply interruption by providing alternative real-time access to IV fluids. These apparatuses, systems, and methods provide a lightweight, smaller footprint and longer shelf-life for IV fluids while still providing the unique and varied types of IV fluids.

The pre-loaded containers can be stacked or stored in a smaller area as compared to IV bags that container an IV fluid. This reduced storage space greatly reduces costs associated therewith.

One embodiment is a unique system, method, and apparatus that includes a pre-loaded container for use in dispensing an IV fluid, the container comprising: a sterile container defining a recess that contains a dry powder composition in a storage configuration, the sterile container further configured to allow for the introduction of a prescribed amount of a sterile water in the recess for agitation with the dry powder composition in a patient dispensing configuration.

In one embodiment, the dry powder composition includes sterile electrolytes and/or dextrose. In one refinement of this embodiment, the sterile electrolytes include one or more of sodium chloride, potassium chloride, magnesium chloride, calcium chloride, potassium phosphate, and potassium acetate. In another refinement of this embodiment, the prescribed amount of the sterile water is 1000 milliliters and the sterile electrolytes include an amount of 50 gm of dextrose or 9 gm sodium chloride. In another refinement of this embodiment, the prescribed amount of the sterile water is 1000 milliliters and the sterile electrolytes include an amount of 50 gm of dextrose and 4.5 gm sodium chloride. In another refinement of this embodiment, the prescribed amount of the sterile water is 1000 milliliters and the sterile electrolytes include an amount of 50 gm of dextrose, 4.5 gm sodium chloride, and 1.5 gm potassium chloride. In another refinement of this embodiment, the prescribed amount of the sterile water is 10 milliliters and the sterile electrolytes include an amount of 90 mg sodium chloride.

In one embodiment, the dry powder composition is made by one or more processes that include sterile filtration, spray drying, lyophilization, and/or gamma radiation.

In one embodiment, the sterile container is an IV bag or a vial.

According to another aspect, a method comprising: providing a sterile container that defines a recess, the recess contains a dry powder composition; inserting a prescribed amount of a sterile water into the recess of the container; and agitating the dry powder composition and the prescribed amount of the sterile water to reconstitute the dry powder composition and the sterile water to form a primary IV fluid.

In one embodiment, the dry powder composition includes sterile electrolytes and/or dextrose. In one refinement of this embodiment, the sterile electrolytes include sodium chloride, potassium chloride, magnesium chloride, calcium chloride, potassium phosphate, potassium acetate.

In one embodiment, the sterile container includes an IV bag or a vial.

In one embodiment, the dry powder composition is made by one or more processes that include sterile filtration, spray drying, lyophilization, and/or gamma radiation.

According to yet another aspect, a method of manufacturing a pre-loaded container for use in dispensing an IV fluid, the method comprising: providing a container that defines a recess sized to receive the IV fluid, the container having an end portion that is configured to open to allow fluid communication with the recess, the container also having an aperture configured to allow for the introduction of the IV fluid to the recess; adding the IV fluid through the aperture to the container; and lyophilizing the IV fluid in the recess of the container to form a dry powder composition in the recess of the container.

In one embodiment, the dry powder composition includes electrolytes and/or dextrose.

In one embodiment, the container includes an IV bag or a vial.

In one embodiment, the lyophilizing the IV fluid further includes: freezing the IV fluid in the container; opening the end portion to vent the recess; and sublimating the frozen IV fluid to form the dry powder composition in the recess of the container.

In one embodiment, the method further comprises inserting a prescribed amount of sterile water into the recess of the container; and agitating the dry powder composition and the prescribed amount of the sterile water to reconstitute the dry powder composition and the sterile water to form a primary IV fluid. In a refinement of this embodiment, the prescribed amount of the sterile water is 1000 milliliters and the dry powder composition includes an amount of one of (i) 50 gm of dextrose, (ii) 9 gm sodium chloride, (iii) 50 gm of dextrose and 4.5 gm sodium chloride, or (iv) 50 gm of dextrose, 4.5 gm sodium chloride, and 1.5 gm potassium chloride.

This summary is provided to introduce a selection of concepts that are further described below in the illustrative embodiments. This summary is not intended to identify key or essential features of the claimed subject matter, nor is it intended to be used as an aid in limiting the scope of the claimed subject matter. Further embodiments, forms, objects, features, advantages, aspects, and benefits shall become apparent from the following description and drawings.

### BRIEF DESCRIPTION OF THE DRAWINGS

The concepts described herein are illustrative by way of example and not by way of limitation in the accompanying figures. For simplicity and clarity of illustration, elements illustrated in the figures are not necessarily drawn to scale. Where considered appropriate, reference labels have been repeated among the figures to indicate corresponding or analogous elements.

FIG. 1 is a front view of a pre-loaded container of the present disclosure.

### DESCRIPTION OF ILLUSTRATIVE EMBODIMENTS

For the purposes of promoting an understanding of the principles of the invention, reference will now be made to the embodiments illustrated in the drawings and specific language will be used to describe the same. It will nevertheless be understood that no limitation of the scope of the invention is thereby intended, any alterations and further modifications in the illustrated embodiments, and any further applications of the principles of the invention as illustrated therein as would normally occur to one skilled in the art to which the invention relates are contemplated herein.

The term, “dry powder formulation” or “dry powder composition” refers to a dry, solid composition of electrolytes, and encompasses dried powder compositions of electrolytes and/or sterile dextrose prepared by lyophilization or freeze-drying, sterile spraying, sterile filtering, gamma radiation, and any other techniques that achieve production of dried electrolytes in a powder form. Other techniques than those listed here may be used to prepare the dry powder formulation or composition. The sterile electrolytes include but are not limited to sodium chloride, potassium chloride, magnesium chloride, calcium chloride, potassium phosphate, potassium acetate, and dextrose includes any type of glucose used for fluid or nutrient replacement.

The present application includes providing a pre-loaded container that includes a sterile container that stores or holds a dry powder composition therein for later use in dispensing an IV fluid. The pre-loaded container with the dry powder composition therein can be stored, shipped, and transported, such that the final product only requires fluid to be added to the sterile container for reconstitution. Upon reconstitution, the resultant solution of reconstituted components converts the fluid and dry powder composition to the primary IV fluid defined by the dry powder composition. A defined or prescribed amount of sterile water for injection (WFI), can be added to the dry powder composition in the sterile container when the primary IV fluid is needed. This invention relieves the burden of variability by simplifying the inventory stored at hospitals, clinics, military, or other locations that use IV fluids. Moreover, the pre-loaded containers are lightweight and reduce the storage space, storage costs, and shipping costs as compared with containers that store IV fluids (IV fluid bag). The pre-loaded containers also have a longer shelf life as compared with containers that store IV fluids. As such, all of the common types of IV fluids can be reconstituted at the site of care using water for injection (WFI) at a fraction of the cost, weight, storage space with a longer shelf life. The pre-loaded containers prior to reconstitution weigh approximately 10% of the IV fluid bags.

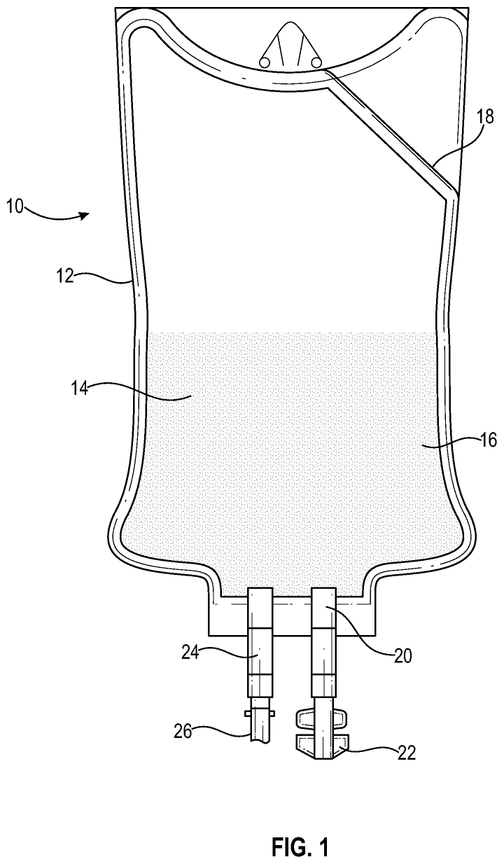

With reference to FIG. 1, a pre-loaded container 10 is illustrated that includes a sterile container 12 that defines a recess 14 within the sterile container 10. The recess 14 contains a dry powder composition 16 within the sterile container 10. In FIG. 1, the sterile container 12 is illustrated in a storage configuration. The sterile container 12 is further configured to accept the introduction of and thereafter contain a prescribed amount of sterile water (not illustrated) in the recess 14 for agitation with the dry powder composition 16 wherein the sterile container 12 is in a patient dispensing configuration. The sterile container 12 in the patient dispensing configuration enables the agitated and reconstituted solution in the recess 14 to be administered intravenously for fluid or nutrient replacement as explained in more detail below.

Some examples of the sterile container 12 include an IV bag, a vial, bottle, or other mechanism that can store the dry powder composition 16 and at a later time enable sterile fluid to be added to the dry powder composition 16. The sterile container 12 includes a first aperture or opening 20 with a removable sealing element 22 thereon that covers the aperture or opening 20. The sealing element 22 can be removed from the opening 20 to accept the introduction of an IV fluid, or a prescribed amount of sterile water into the recess 14 of the container 20 for agitation with the dry powder composition 16 in the recess 14. Optionally, drugs can also be added through the opening 20 into the recess 14.

The sterile container 12 includes an end portion 18 that is opposite to the aperture or opening 20. The end portion 18 can be opened to allow for addition of the dry powder composition 16, sterile fluid or water, or any other IV fluid into the recess 14. Additionally, the end portion 18 can be opened to allow the recess 14 to be ventilated as described more below. For example, during lyophilization or other processes in which the dry powder composition 16 is formed in the sterile container 12, it is necessary to allow the recess 14 to ventilate and allow water crystals to sublimate under vacuum pressure to form the dry powder composition 16. The end portion 18 can be re-sealed and closed after the formation of the dry powder composition 16. In the illustrated embodiment, the end portion 18 is positioned at a corner of the sterile container 12. In other embodiments, the end portion 18 extends across a width of the sterile container 12.

In some forms, the sterile container 12 includes a second aperture or opening 24 for receipt of a tube configured as an IV line 26 to allow for the delivery of the IV fluid to a patient. If the IV line 26 is not assembled with the opening 24, then a second sealing element (not illustrated) would seal the second opening 24 such that the dry powder composition 16 is retained in the recess 14.

The pre-loaded container 10 includes the dry powder composition 16 in the recess 14. Pre-measured amounts of the dry powder composition 16 include 50 gm of dextrose; 9 gm sodium chloride; 50 gm of dextrose and 4.5 gm sodium chloride; 50 gm of dextrose, 4.5 gm sodium chloride, and 1.5 gm potassium chloride; or 90 mg sodium chloride. These pre-measured amounts of the dry powder composition 16 enable a medical professional to add a prescribed amount of the sterile fluid such as 1000 milliliters of WFI to the aperture 20 to enable agitation and reconstitution of the dry powder composition 16 to form a resultant or primary IV fluid for use by a medical patient. For example, the resultant or primary IV fluids formed from the above listed dry powder compositions 16 are, respectively, dextrose 5%/water 1000 mL; 0.9% sodium chloride 1000 mL or normal saline (NS); dextrose 5%/0.45% NS 1000 mL; dextrose 5%/0.45% NS+20 mEq KCl 1000 mL; and 0.9% sodium chloride 10 mL or IV line flush.

Some of the techniques that can be used to insert the dry powder composition 16 into the sterile container 12 include sterile spraying, sterile filtering, gamma radiation, and/or lyophilization, and any combination of these techniques may be used. Any of these processes or other processes not listed can be used to produce micronized material (high surface area) for sterile, particle free use. Some techniques that can be used to form the dry powder composition 16 in the container 12 include but are not limited to sterile filtration of the IV fluid, sterile crystallization, sterile micronization, and then the dry powder composition 16 is aseptically bagged for use in the sterile container 12. Another technique includes sterile filtration or particle removal of the IV fluid, followed by clean crystallization, clean micronization, packaging of the dry powder composition 16 in the sterile container 12 and gamma irradiation or e-beam sterilization of the dry powder composition 16. Yet another technique includes sterile filtration of the IV fluid, sterile spray drying of the IV fluid, and then the dry powder composition 16 is aseptically bagged for use in the sterile container 12. A further technique includes sterile filtration of the IV fluid, sterile freeze-drying of the IV fluid, and then the dry powder composition 16 is aseptically bagged for use in the sterile container 12. Yet another technique includes sterile filtration or particle removal of the IV fluid, clean freeze-drying of the IV fluid, packaging the dry powder composition 16 in the sterile container 12, and then gamma irradiation or e-beam sterilization of the dry powder composition 16. Still yet another technique includes sterile filtration or particle removal of the IV fluid, clean spray drying of the IV fluid, packaging the dry powder composition 16 in the sterile container 12, and then gamma irradiation or e-beam sterilization of the dry powder composition 16. One or more of these techniques can be combined to form the dry powder composition 16 in the container 12.

Yet another technique of placing or forming the dry powder composition in the recess 14 of the sterile container 12 includes lyophilization which is generally a process of freeze-drying in which water is sublimed from the product after it is frozen, optionally by applying a vacuum. The lyophilization process generally includes the following steps. First, dissolving the drug and excipients in a suitable solvent, generally water for injection (WFI), to create a bulk solution. Next, the bulk solution is sterilized by passing it through a 0.22 micron bacteria-retentive filter. Next, appropriate amounts of the sterilized bulk solution is added into individual sterile containers 12 and the apertures 20 are closed or sealed with the sealing element 22 under aseptic conditions.

The containers 12 are transferred to a lyophilizer and loaded into the chamber of the lyophilizer under aseptic conditions. Next, the solution in the sterile containers 12 is frozen by placing the containers 12 on cooled shelves in a freeze-drying chamber or pre-freezing in another chamber. The end portion 18 is opened to allow ventilation of the recess 14. Next, a vacuum is applied to the chamber and the shelves are heated in order to evaporate the water from the frozen state which leaves the dry powder composition 16 in the recess 14 of the container 12 to form the pre-loaded container 10. In one form, the end portion 18 is sealed or closed while the sterile container 12 is within the lyophilizer. In an alternate form, the sterile container 12 is removed from the lyophilizer and the end portion 18 is sealed.

One example of forming the pre-loaded container 10 with the dry powder composition 16 including 9 gm sodium chloride includes the following steps. First a concentrated solution of 9 gm of sodium chloride in 23.01 mL of WFI that forms a bulk solution is added to the sterile container 12. The sterile container 12 with the bulk solution in the recess 14 is loaded into lyophilizer at room temperature. Next, the container 12 is cooled to −40° C. at rate of 0.5° C. per minute and then held at −40° C. for 1 hour. The end portion 18 is opened Next a vacuum is set to 100 m Torr and the container 12 is warmed to −15° C. and then held at −15° C. and 100 mTorr for 36 hours. Next, the sterile container 12 is warmed to 30° C. at rate of 1° C./minute and held at 30° C. for 90 minutes to evaporate any fluid. As such, the dry powder composition 16 including 9 gm sodium chloride remains in the recess 14 of the container 12 to form the pre-loaded container 10. Finally, the end portion 18 is sealed and the sterile container 12 is removed from the lyophilizer.

The process of adding the lyophilized electrolyte components or dry powder composition 14 is achieved by placing a maximally concentrated solution of the electrolytes into the sterile container 12 and then undergoing the lyophilization process. Examples of the solutions are shown in the table below.

| | Final Volume after | Required Amount of | Concentrated Solution to be |
|---|---|---|---|
| Final Concentration | Reconstitution | Component | added to empty bag* |
| 5% w/v Dextrose | 1000 mL | 50 grams Dextrose | 50 gm Dextrose/55.2 mL water |
| 0.9% w/v NaCl | 1000 mL | 9 grams NaCl | 9 gm NaCl/23.01 mL water |
| 0.45% w/v NaCl | 1000 mL | 4.5 grams NaCl | 4.5 gm NaCl/11.5 mL water |

To prepare a primary IV fluid for use by a medical patient, the medical professional would insert a prescribed amount of a single sterile fluid such as sterile WFI into the recess 14 of the pre-loaded container 10. The medical professional would mix or agitate the dry powder composition 16 and the prescribed amount of the sterile water for injection to reconstitute the dry powder composition 16 and the sterile water to form a primary IV fluid.

The prescribed amount of sterile water for injection (WFI) can be formed in a variety of ways. For example, many hospital pharmacies have an IV repeater pump that can be used to transfer sterile WFI to the pre-loaded container 10. An IV repeater pump insures the correct or prescribed amount of sterile WFI is accurately transferred to the recess 14 of the pre-loaded container 16.

The use of pre-loaded containers 16 reduce the medical professionals' inventory of bulk IV fluids in bags or vials. The medical professionals need only procure sterile WFI or produce sterile WFI independently using a machine that produces WFI via reverse osmosis. For example, some machines are capable of producing 4-11 liters per minute of WFI, and during continuous operation, 12,600 liters per day. This greatly exceeds the average hospital daily utilization.

As is evident from the figures and text presented above, a variety of aspects of the present disclosure are contemplated.

Various aspects of the present application are contemplated. According to one aspect, a pre-loaded container for use in dispensing an IV fluid, the container comprising: a sterile container defining a recess that contains a dry powder composition in a storage configuration, the sterile container further configured to allow for the introduction of a prescribed amount of a sterile water in the recess for agitation with the dry powder composition in a patient dispensing configuration.

In one embodiment, the dry powder composition includes sterile electrolytes and/or dextrose. In one refinement of this embodiment, the sterile electrolytes include one or more of sodium chloride, potassium chloride, magnesium chloride, calcium chloride, potassium phosphate, and potassium acetate. In another refinement of this embodiment, the prescribed amount of the sterile water is 1000 milliliters and the sterile electrolytes include an amount of 50 gm of dextrose or 9 gm sodium chloride. In another refinement of this embodiment, the prescribed amount of the sterile water is 1000 milliliters and the sterile electrolytes include an amount of 50 gm of dextrose and 4.5 gm sodium chloride. In another refinement of this embodiment, the prescribed amount of the sterile water is 1000 milliliters and the sterile electrolytes include an amount of 50 gm of dextrose, 4.5 gm sodium chloride, and 1.5 gm potassium chloride. In another refinement of this embodiment, the prescribed amount of the sterile water is 10 milliliters and the sterile electrolytes include an amount of 90 mg sodium chloride.

In one embodiment, the dry powder composition is made by one or more processes that include sterile filtration, spray drying, lyophilization, and/or gamma radiation.

In one embodiment, the sterile container is an IV bag or a vial.

According to another aspect, a method comprising: providing a sterile container that defines a recess, the recess contains a dry powder composition; inserting a prescribed amount of a sterile water into the recess of the container; and agitating the dry powder composition and the prescribed amount of the sterile water to reconstitute the dry powder composition and the sterile water to form a primary IV fluid.

In one embodiment, the dry powder composition includes sterile electrolytes and/or dextrose. In one refinement of this embodiment, the sterile electrolytes include sodium chloride, potassium chloride, magnesium chloride, calcium chloride, potassium phosphate, potassium acetate.

In one embodiment, the sterile container includes an IV bag or a vial.

In one embodiment, the dry powder composition is made by one or more processes that include sterile filtration, spray drying, lyophilization, and/or gamma radiation.

According to yet another aspect, a method of manufacturing a pre-loaded container for use in dispensing an IV fluid, the method comprising: providing a container that defines a recess sized to receive the IV fluid, the container having an end portion that is configured to open to allow fluid communication with the recess, the container also having an aperture configured to allow for the introduction of the IV fluid to the recess; adding the IV fluid through the aperture to the container; and lyophilizing the IV fluid in the recess of the container to form a dry powder composition in the recess of the container.

In one embodiment, the dry powder composition includes electrolytes and/or dextrose.

In one embodiment, the container includes an IV bag or a vial.

In one embodiment, the lyophilizing the IV fluid further includes: freezing the IV fluid in the container; opening the end portion to vent the recess; and sublimating the frozen IV fluid to form the dry powder composition in the recess of the container.

In one embodiment, the method further comprises inserting a prescribed amount of sterile water into the recess of the container; and agitating the dry powder composition and the prescribed amount of the sterile water to reconstitute the dry powder composition and the sterile water to form a primary IV fluid. In a refinement of this embodiment, the prescribed amount of the sterile water is 1000 milliliters and the dry powder composition includes an amount of one of (i) 50 gm of dextrose, (ii) 9 gm sodium chloride, (iii) 50 gm of dextrose and 4.5 gm sodium chloride, or (iv) 50 gm of dextrose, 4.5 gm sodium chloride, and 1.5 gm potassium chloride.

Reference throughout this specification to “one embodiment,” “an embodiment,” or similar language means that a particular feature, structure, or characteristic described in connection with the embodiment is included in at least one embodiment of the present disclosure. Appearances of the phrases “in one embodiment,” “in an embodiment,” and similar language throughout this specification may, but do not necessarily, all refer to the same embodiment. Similarly, the use of the term “implementation” means an implementation having a particular feature, structure, or characteristic described in connection with one or more embodiments of the present disclosure, however, absent an express correlation to indicate otherwise, an implementation may be associated with one or more embodiments.

The described features, structures, advantages, and/or characteristics of the subject matter of the present disclosure may be combined in any suitable manner in one or more embodiments and/or implementations. In the following description, numerous specific details are provided to impart a thorough understanding of embodiments of the subject matter of the present disclosure. One skilled in the relevant art will recognize that the subject matter of the present disclosure may be practiced without one or more of the specific features, details, components, materials, and/or methods of a particular embodiment or implementation. In some instances, the benefit of simplicity may provide operational and economic benefits and exclusion of certain elements described herein is contemplated as within the scope of the invention herein by the inventor to achieve such benefits. In other instances, additional features and advantages may be recognized in certain embodiments and/or implementations that may not be present in all embodiments or implementations. Further, in some instances, well-known structures, materials, or operations are not shown or described in detail to avoid obscuring aspects of the subject matter of the present disclosure. The features and advantages of the subject matter of the present disclosure will become more fully apparent from the following description and appended claims, or may be learned by the practice of the subject matter as set forth hereinafter.

The present subject matter may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. A pre-loaded container for use in dispensing an IV fluid, the container comprising:
a sterile container defining a recess that contains a dry powder composition in a storage configuration, the sterile container further configured to allow for the introduction of a prescribed amount of a sterile water in the recess for agitation with the dry powder composition in a patient dispensing configuration.

2. The apparatus of claim 1, wherein the dry powder composition includes sterile electrolytes and/or dextrose.

3. The apparatus of claim 2, wherein the sterile electrolytes include one or more of sodium chloride, potassium chloride, magnesium chloride, calcium chloride, potassium phosphate, and potassium acetate.

4. The apparatus of claim 2, wherein the prescribed amount of the sterile water is 1000 milliliters and the sterile electrolytes include an amount of 50 gm of dextrose or 9 gm sodium chloride.

5. The apparatus of claim 4, wherein the prescribed amount of the sterile water is 1000 milliliters and the sterile electrolytes include an amount of 50 gm of dextrose and 4.5 gm sodium chloride.

6. The apparatus of claim 4, wherein the prescribed amount of the sterile water is 1000 milliliters and the sterile electrolytes include an amount of 50 gm of dextrose, 4.5 gm sodium chloride, and 1.5 gm potassium chloride.

7. The apparatus of claim 4, wherein the prescribed amount of the sterile water is 10 milliliters and the sterile electrolytes include an amount of 90 mg sodium chloride.

8. The apparatus of claim 1, wherein the dry powder composition is made by one or more processes that include sterile filtration, spray drying, lyophilization, and/or gamma radiation.

9. The apparatus of claim 1, wherein the sterile container is an IV bag or a vial.

10. A method comprising:
providing a sterile container that defines a recess, the recess contains a dry powder composition;
inserting a prescribed amount of a sterile water into the recess of the container; and
agitating the dry powder composition and the prescribed amount of the sterile water to reconstitute the dry powder composition and the sterile water to form a primary IV fluid.

11. The method of claim 11, wherein the dry powder composition includes sterile electrolytes and/or dextrose.

12. The method of claim 12, wherein the sterile electrolytes include sodium chloride, potassium chloride, magnesium chloride, calcium chloride, potassium phosphate, potassium acetate.

13. The method of claim 11, wherein the sterile container includes an IV bag or a vial.

14. The method of claim 11, wherein the dry powder composition is made by one or more processes that include sterile filtration, spray drying, lyophilization, and/or gamma radiation.

15. A method of manufacturing a pre-loaded container for use in dispensing an IV fluid, the method comprising:
providing a container that defines a recess sized to receive the IV fluid, the container having an end portion that is configured to open to allow fluid communication with the recess, the container also having an aperture configured to allow for the introduction of the IV fluid to the recess;
adding the IV fluid through the aperture to the container; and
lyophilizing the IV fluid in the recess of the container to form a dry powder composition in the recess of the container.

16. The method of claim 15, wherein the dry powder composition includes electrolytes and/or dextrose.

17. The method of claim 15, wherein the container includes an IV bag or a vial.

18. The method of claim 15, wherein the lyophilizing the IV fluid further includes:
freezing the IV fluid in the container;
opening the end portion to vent the recess; and
sublimating the frozen IV fluid to form the dry powder composition in the recess of the container.

19. The method of claim 15, further comprising:
inserting a prescribed amount of sterile water into the recess of the container; and
agitating the dry powder composition and the prescribed amount of the sterile water to reconstitute the dry powder composition and the sterile water to form a primary IV fluid.

20. The method of claim 19, wherein the prescribed amount of the sterile water is 1000 milliliters and the dry powder composition includes an amount of one of (i) 50 gm of dextrose, (ii) 9 gm sodium chloride, (iii) 50 gm of dextrose and 4.5 gm sodium chloride, or (iv) 50 gm of dextrose, 4.5 gm sodium chloride, and 1.5 gm potassium chloride.
